(19) United States
(12) Patent Application Publication
(10) Pub. No.: US 2010/0004132 A1
(43) Pub. Date: Jan. 7, 2010

(54) MODIFIED INORGANINC MATERIALS

(75) Inventors: Cameron John Kepert, Lillyfield (AU); Malcolm Edward Roland Green, New South Wales (AU); Sarah Jane Antill, New South Wales (AU)

Correspondence Address: WOLF GREENFIELD & SACKS, P.C., 600 ATLANTIC AVENUE, BOSTON, MA, 02210-2206, US

(73) Assignee: THE UNIVERSITY OF SYDNEY, New South Wales (AU)

(21) Appl. No.: 11/989,467

(22) PCT Filed: Jul. 26, 2006
(86) PCT No.: PCT/AU2006/001050

§ 371 Date: Jul. 23, 2009

(30) Foreign Application Priority Data
Jul. 26, 2005 (AU) 2005903969

(51) Int. Cl. A01N 25/08; B05D 7/22; C05D 9/00; A61K 9/00; A61K 8/02; C11D 17/00; C02F 5/08; C09K 15/00; A01N 37/18; A01N 33/18; A01P 1/00; A01P 13/00

(52) U.S. Cl. 504/340; 427/230; 427/235; 427/595; 427/238; 71/62; 504/359; 424/409; 424/400; 512/4; 510/438; 252/175; 252/387; 504/347

(57) ABSTRACT

A method of conditioning a tubular clay material to enable its loading with an active, the method comprising the step of exposing the tubular clay material to a chemical agent in a manner such that the agent sorbs to a surface of the clay material that is internal of the tube, the chemical agent being selected such that, when the agent is sorbed to the clay material internal surface, the affinity of the tubular clay material for the active is altered.

### TECHNICAL FIELD

Methods for modifying tubular shaped inorganic materials are disclosed, as are the resultant materials themselves. More particularly the method involves the conditioning of a tubular clay material to alter its affinity for an active chemical (hereafter “active”).

### BACKGROUND ART

Global demand for inexpensive technologies that allow the controlled release of actives such as herbicides, pesticides and fertilizers is increasing. In particular, there is a demand for controlled release hosts (or carriers) that are inexpensive, have higher weight-per-weight loadings, are environmentally friendly, have release rates that can be controlled to range from days to months, and that can act as hosts for a range of actives that have a biological effect.

Attempts to meet these criteria at the higher expense end of the market have involved micro-encapsulation of actives such as agrochemicals into polymer-based granules and spheroids. Examples include products marketed under the trade names “SusCon” for use with sugarcane and “Osmocote” for general horticultural use. Developments at the lower expense end have been limited. As a result, large-scale delivery of agrochemicals currently occurs with great inefficiencies.

U.S. Pat. No. 5,651,976 discloses a composition and method for delivering an active agent at a controlled rate. The patent discloses a hollow ceramic or inorganic microtubule wherein an active agent is adsorbed onto an inner surface of a lumen of the microtubule. The microtubule “loaded” with active agent is then further treated to provide for a subsequent controlled release.

### SUMMARY

In a first aspect there is provided a method of conditioning a tubular clay material to enable its loading with an active, the method comprising the step of exposing the tubular clay material to a chemical agent in a manner such that the agent sorbs to a surface of the clay material that is internal of the tube, the chemical agent being selected such that, when the agent is sorbed to the clay material internal surface, the affinity of the tubular clay material for the active is altered.

The method of conditioning the tubular clay material occurs prior to loading it with an active and can be used to enhance and optimise the manner by which the material performs with the active in use. The conditioned tubular clay can be used to absorb substances for removal or collection or to allow for loading with an active and controlled release of an active

The tubular clay material is typically one or more of halloysite, imogolite, boulangerite and cylindrite, each of which naturally form nanotubes. The term “tubular” includes untapped tubular forms and lapped (e.g. “carpet roll”) tubular forms.

The chemical agent can be one or more of a surfactant, an alcohol and a phosphonate. In each case the agent can bond to the clay material internal surface to be sorbed thereto, whereby the internal surface properties can then be changed (e.g. to render the lumen with a greater affinity for an active). This can enhance the variety and extent of actives that can be loaded into the clay material, as compared to previous approaches.

When the agent is a surfactant, the step of exposing the tubular clay material to the surfactant can involve refluxing a solution of the material with the surfactant. For example, the material can be suspended in an aqueous solution of the surfactant and refluxed. Refluxing to condition the clay material may only require a temperature of 80° C. for one hour. After refluxing, the solution can be cooled, filtered and washed to remove residual surfactant.

Further, the ratio (w/w) of tubular clay material to surfactant can range from 1:1 to 1:20, depending on the active and the application.

Because the typical tubular clay materials employed are anionic, the surfactant is typically cationic. It may be selected from one or more of:
alkylammonium surfactants such as hexadecyl-trimethyl ammonium (HDTMA) and octyl-trimethylammonium (OTMA);
phenylammonium surfactants such as benzyl-trimethylammonium (BTMA) and phenyl-trimethylammonium PTMA);
substituted phenylammonium surfactants;
alkylpyridinium surfactants; and
phenylpyridinium surfactants.

When the agent is an alcohol, the step of exposing the tubular clay material to the alcohol can involve mixing the clay material with the alcohol and heating the mixture so as to promote a condensation reaction between the alcohol and the clay material at the internal surface, thus forming a strong bond.

To promote the condensation reaction, the mixture may first be heated using microwave irradiation and may subsequently be refluxed. During microwave irradiation and prior to refluxing, a vacuum can applied to the alcohol and clay material mixture to remove air from the clay material tubes.

In one embodiment the alcohol selected is one that refluxes at a temperature just below its boiling point. Thus, the alcohol can first be control heated to its reflux temperature using microwave irradiation. For example, the alcohol can be 1-octanol, with the mixture being heated to 194° C. using microwave irradiation and then being refluxed at 194° C. for up to 56 hours to ensure complete reaction.

When the agent is a phosphonate, the step of exposing the tubular clay material to the phosphonate can involve mixing the clay material with the phosphonate and heating the mixture so as to promote a reaction between the phosphonate and the clay material at the internal surface.

Again the mixture can be heated using microwave irradiation. Further, prior to heating the mixture, microwave irradiation, slow heating or vacuum cycling can be applied to the phosphonate and clay material mixture to remove air from the clay material tubes. After heating using microwave irradiation, the mixture can be washed with dichloromethane and methanol to remove residual phosphonate.

The phosphonate can be a phosphonate ester such as diethyl phosphonate or diethyl benzyl-phosphonate.

After the tubular clay material has been conditioned, the material may more optimally be subjected to loading with an active. The method of loading employed may then be an active-melting loading technique or an active-in-solution loading technique.

In the active-melting loading technique, the active and clay material can be mixed and then heated to a temperature above the melting point for the active, and held there for a time period sufficient for the active to migrate into the tube (e.g. via capillary action). The time period may be at least 5 hours.

In the active-in-solution loading technique, the active can be dissolved in a solution in which it is soluble (usually very soluble) and the clay material can then be added to this solution, either with the active or after, and the solution then stirred.

The stirred solution can then be subjected to ultrasound, subjected to a vacuum, centrifuged to remove supernatant solution, and then dried. For example, the loaded clay material can be dried at 90° C. for 24 hours to produce a ready-to-use loaded (or charged) clay material.

The active can comprise one or more inorganic and one or more organic chemicals, including mixtures thereof. For example, the active can be one or more of an agrochemical, a pharmaceutical, a biocide, a bactericide, an anti-foulant, a cosmetic, a fragrance, a detergent, a hormone, a pheromone, a descaler, a cleaning agent, a bactericide, a corrosion inhibitor/preventer, an organic or inorganic pollutant or toxic material. The agrochemical can be one or more of alochlor, metachlor and trifluralin. This diversity of actives indicates the diversity of applications of the conditioned clay material.

The conditioning employed may then be targeted or tuned to the specific active and its application. For example, the affinity of the tubular clay material for the active can be altered in a manner such that the material can act to release active in a controllable manner and/or to store or entrap active. In other words, the application of the conditioned clay material is not limited to controlled release, and the clay material may be used for clean up and thus safe handling and storage of e.g. toxic wastes and spills. In addition, the controlled release can be tuned through the careful selection of one or more of the chemical agents initially loaded into the clay material.

Further, the tube length and/or tube length distribution can be varied prior to loading of chemical agent and active. For example, the tube length and/or tube length distribution can be varied by one or more of: tube sourcing control, milling and centrifuging.

In a second aspect a tubular clay material is provided that has been conditioned to enable its loading with an active. The clay material comprises a chemical agent that is sorbed to a surface of the material that is internal of the tube and that alters the material's affinity for the active.

The tubular clay material of the second aspect can be as defined in, and as conditioned by, the method of the first aspect.

The conditioned tubular clay material can be utilised for uptake and removal of actives such as wastes and toxic substances. That is the conditioned tubular clay material can act to release active in a controllable manner and/or to store or entrap active for removal of actives.

### BRIEF DESCRIPTION OF DRAWINGS

Notwithstanding any other forms that may fall within the scope of the method and tubular clay material as defined in the Summary, specific embodiments of the method and material will now be described, by way of example only, with reference to the accompanying drawings and Examples in which:

FIG. 1 schematically depicts a halloysite tube showing possible locations for load of active;

FIG. 2 shows four separate TEM images of halloysite tubes; and,

FIG. 3 plots isotherms for modified and unmodified halloysite.

### DETAILED DESCRIPTION OF SPECIFIC EMBODIMENTS

In this description the terminology “controlled release” is used to refer to systems that are able to release actives from within their structures at desired and/or controllable rates. Controlled release is desirable because it allows maintenance of appropriate dosing without frequent delivery and/or initial overdosage. It can also reduce levels of exposure during handling. Controlled release has a wide range of applications, including, but not limited to, the delivery of pharmaceuticals, cosmetics, fragrances, detergents and agrochemicals.

The tubular clays employed herein (sometimes also referred to as “cylindrical” clays) when acting as controlled release hosts had an advantage over other clays because the inside (“lumen”) of the tubes was able to be used to encapsulate a greater amount of active than surface area alone allowed (e.g. as compared with a generally layered clay material). Additionally, the one-dimensional diffusion out of the lumen offered the opportunity to obtain desired release rates by manipulating the interactions between the modified clay and the active through the selection of appropriate conditioning modifications for each active.

Tubular clay materials employed included halloysite, imogolite, boulangerite and cylindrite. The tubes could comprise an untapped wall or be in a lapped (or “carpet roll”) form.

To achieve low cost controlled release the clays were first modified (conditioned) as carriers. For example, cationic surfactants were adsorbed onto the clay internal surface, with an active (e.g. an organic agrochemical) then able to be sorbed to the non-polar tail of the surfactant. This decreased the mobility of the active and allowed for control over its rate of release. Additionally, the capacity of the clay to carry the active (the % load) was increased due to conditioning and the overall increased surface area available with tubular clays.

Further, the conditioning of the tubular clay material widened the range of hydrophobicities of actives that could be loaded into tube lumen 12. It also improved the degree of control over release rates, as the choice of clay modification was able to be used to control the rate of diffusion out of the tubes. This was achieved in two ways:
long chain organic agents were sorbed to the internal tube walls, decreasing the internal tube diameter and thus the rate and total amount of active diffusing out of the tube;
the functional groups and chain lengths of the modifying chemical agent was selected to promote optimal interaction with the active, allowing further control over diffusion out of the tube.

Methods were also investigated for controlling the rate of release of active by end-capping the tubes, by modifying tube length, and by coating the tubes with porous polymer or biodegradable binder.

The tubular clay materials were capable of receiving a high percentage load for a wide range of active compounds and were sufficiently inexpensive to allow application on e.g. an agricultural scale. Thus, an important application was in the controlled release of agrochemicals on a large (multi-field) scale. Examples of agrochemical actives that were used included alochlor, metachlor and trifluralin. However, other potential applications included:
controlled release in pharmaceuticals;
cosmetics;
skin care, for example mud packs;
paints incorporating biocides and antifouling agents;
deliveries to subsurface oil and gas reserves/water reservoirs of descalers and chemical cleaners, bactericides and corrosion inhibitors/preventers.

For each of these applications the tubular clay materials offered high load ratios, an ability to act as carriers for a wide range of compounds, release “tunability”, and an ability to encapsulate toxic actives within the lumen 12.

### High Load Ratios

Lumen 12 load capability of unmodified halloysite was observed to be 44% v/v, with potentially higher w/w ratios for actives of specific gravity greater than 2.55 g/cm³. This was in addition to the exogenous and interlayer loads which, by geometry, were comparable to the total load of flat clays. The enhanced load ratio resulting from conditioning allowed smaller amounts of carrier to be used in each controlled release application. Consequently production, handling and transport costs were reduced.

### Wide Range of Compounds and Release Rate “Tuning”

Design of the host-guest (chemical agent/active) interaction was also investigated to allow a wider range of compounds to be loaded than on an modified halloysite. This was directly applicable to controlled release application, because the favourable interactions between host and guest resulted in an inherently slower release. Further, judicious choice of the chemical agent/active combination allowed control of release patterns.

### Encapsulation of Compounds

The encapsulation of various poisonous agrochemicals and toxic materials inside the lumen 12 of the tubular halloysite was beneficial, as handling of the chemicals was made safer (i.e. there was no contact between the handler and toxic chemical).

A most desirable outcome was release rates ranging from days to months, to optimise uniform dosage.

### EXAMPLES

Whilst the modification (conditioning of inorganic tubes included modifying halloysite, imogolite, boulangerite and cylindrite, and involved loading and controlling of release of actives, the tubular clay halloysite was initially used as a demonstration example.

Halloysite (Al₂(OH)₄Si₂O₅.2H₂O— also known as endellite) is a hydrated polytype of the 1:1 phyllosilicate clay kaolinite. Closely related phases include metahalloysite, Al₂(OH)₄Si₂O₅, obtained by dehydration, and the very rare hydrohalloysite Al₂(OH)₄Si₂(O,OH)₅. Its tubular structure is due to the warping of the clay layers which, under favourable geological conditions, wrap around on themselves cylinders.

### Example 1

Halloysite was obtained from a test site in Camel Lake, South Australia and from New Zealand China Clay Ltd of grade G. The characteristics of these halloysites were determined and are as shown in Table 1:

| Type | Halloysite G (HG) | Camel Lake (Cla1) |
|---|---|---|
| Length (μm) | 0.5 | 0.85 |
| External Diameter (nm) | 30 | 60 |
| Internal Diameter (nm) | 23 | 24.5 |
| Calculated Aspect Ratio | 6.00 × 10 − 2 | 7.06 × 10 − 2 |
| Specific Surface Area (m2/g) | 65.1 | 25.3 |
| Lumen Volume (cm3/g) | 0.16 | 0.075 |

### Example 2

### Clay Conditioning

Three methods were used to modify (condition) the clays of Example 1, in this case, the halloysite tubes:

(a) Surfactant Modification:
Halloysite (5.0 g) was refluxed (30 min, 80° C.) with water (500 ml). HDTMA (8 ml, HDTMA aqueous solution 25% grade in H₂O, Fluka) was added and the suspension refluxed (1 hr, 80° C.), then allowed to cool overnight with stirring. The suspension was filtered and washed with water until the counter ion was not detected in the filtrate by one drop of AgNO3 (0.1 M. The ratio (w/w) of clay to HDTMA was also varied to control the extent of modification. The ratios used were 1:1, 2:3, 1:5, 1.10 and 1:20.

(b) Alcohol Modification:
Halloysite (dried and ground) was placed into a vacuum tube and covered with 1-octanol. The mixture was sired and heated to 194° C. using microwave irradiation. A vacuum was applied to the mixture until the appearance of “fizzing” stopped (i.e. the air trapped in the tubes had escaped). Atmospheric pressure was reapplied and the mixture was refluxed at 194° C. for 56 hours.
This method was not limited to 1-octanol, and was applied to other alcohols, typically those with a reflux temperature of just below the respective boiling point.

(c) Phosphonate Modification:
A phosphonate ester (examples diethyl ethylphosphonate and diethyl benzylphosphonate) (1 mL) was droppered onto halloysite tubules (520 mg) and the air in the tubes was removed by slow heating or vacuum cycling. The mixture was radiated by microwaves (15×4 min) and washed with dichloromethane and methanol to remove residual phosphonate.

### Example 3

### Loading of Conditioned Clay

For actives of low melting point, the “melt method” of loading was used. For actives of high melting point, the “solution method” was used.

### Melt Method:

Dried modified clay was ground in a 1:1 (w/w) ratio with the active to be loaded. The mix was heated to above the melting point of the active for 5 hours. The active entered the lumen 12 via capillary action.

### Solution Method:

A solvent was selected in which the active was highly soluble. For example, to load the compound benzophenone, acetone was used. A saturated solution of active was mixed with dried, ground modified clay by stirring, followed by exposure to an ultrasonic bath (20 min). The mix was placed in a Schlenk tube and subjected to three vacuum cycles, and was then centrifuged. After removal of supernatant solution, the loaded clay was dried at 90° C. for 24 hours.

The modes of delivery of loaded modified tubes differed between applications. This example also outlined a method of delivery devised for agrochemical actives. Loaded modified tubes were able to be used as so produced, or were able to be pelletised with or without a biodegradable and/or water soluble binder. Pelletisation permitted easier transport and handling, as spills were easier to contain and tubes were not lost to air as dust. Loaded modified tubes, or pelletisations with no binder, or combinations with a water soluble binder, were able to be used in spray applications to fields, crops etc.

### Example 4

### Calculation of Diffusion Rates

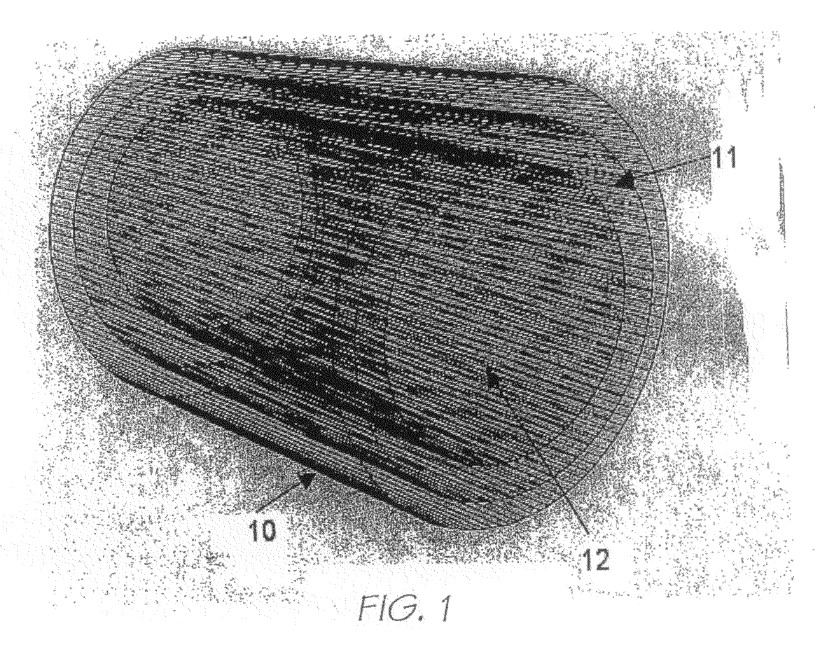

Referring to FIG. 1, a schematic of a halloysite tube shows possible locations for load of active: A) outside surface 10; B) interlayer 11; and C) lumen 12. Delayed release thus occurred from three locations in the halloysite, and each could now be controlled.

Release from the external surface was due to desorption. Release from the interlayer 11 was slower than desorption from the surface, as molecules interacted with the clay layers as they diffused through the interlayer 11. Release from the lumen 12 was governed by a standard equation for one dimensional diffusion, regulated by interactions with the modified inner surface of the tubes.

Release from the lumen 12 was the main focus for controlled release. However, using the variable release rates of active from erogenous and interlayer load allowed an extra degree of flexibility and control over release patterns.

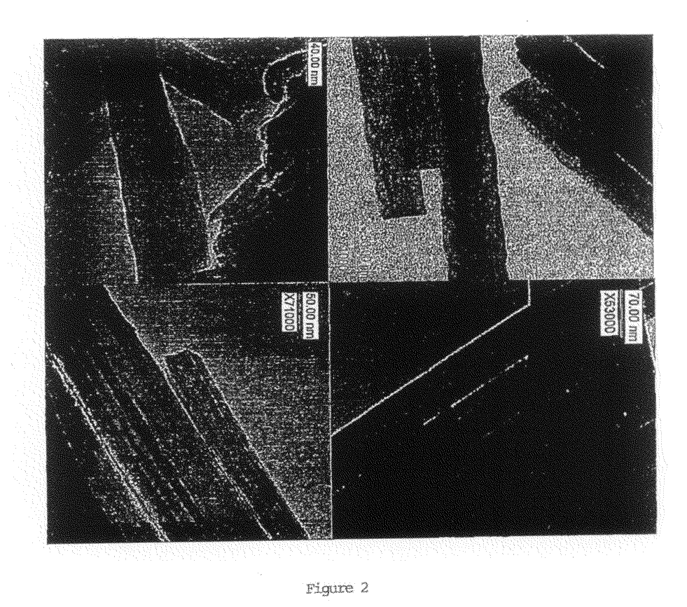

FIG. 2 shows TEM images of halloysite tubules as follows: a) unmodified halloysite with benzophenone adhering to the outside of the tubules; b) modified halloysite with benzophenone only in the tubules; c) unmodified halloysite with 3-aminophenol only on the outside of the tubules; d) modified halloysite with 3-aminophenol inside the tubules.

Using the dimensions obtained from the TEM images of FIG. 2 the active/carrier volume/volume load ratio was calculated to be 44%. The following equations apply:

$$\begin{matrix}volume \text{/} volume load = \frac{lumen volume}{total tube volume} \\ = \frac{0.25 \times length \times II \times {( internal diameter )}^{2}}{0.25 \times length \times II \times {( external diameter )}^{2}} \\ = \frac{{( 20 nm )}^{2}}{{( 30 nm )}^{2}} \\ = 44 \%\end{matrix}$$

For those many actives with a density greater than that of halloysite (2.55 g/cm³), the w/w load was greater than 44%.

The one-dimensional diffusion equation can be written as follows:

$$\frac{d {m}_{lumen}}{d t} = DeffA ρ \{ \frac{{C}_{max} - {C}_{b}}{M + [ {\int }_{{t}_{0}}^{ } ( dm / dt ) d t ) ( A ρ ) ]} \}$$

where m is the mass of active inside the tube;

t is time; D_{eff} is the effective diffusion coefficient of the active in the release solvent (for agrochemical applications, this would be water) regulated by interactions with the modified lumen walls;

A is the cross-sectional area of the tube;

ρ is the density of the active;

Cₘₐₓ is the solubility of the active in the release solvent;

C_{b} is the concentration of the active in the bulk release solvent (assumed insignificantly different from zero); and

M is the total mass of active originally loaded.

Release from the lumen was regulated by changing any of these variables. The choice of loaded active controlled D_{eff}, ρ, M and Cₘₐₓ. The choice of chemical agent (e.g. organo-modification) controlled most significantly D_{eff}, but also A and consequently M. The engineering of the active/modification combination to control release rate over time, with particular emphasis on selection of functional groups, branching and chain lengths, was therefore achievable and delivered refinable (tuneable) controlled release.

It was also observed that lowering D_{eff} by increasing the interaction between the modification functional group and the loaded active was analogous to increasing the shear at the interface between a pipe and a fluid flowing through it. Dissolution of active at the sides of the tube was slowed and, once dissolved, the active was able to resorb out of solution back onto the inner tube walls, further slowing release. The active at the centre of the tube was affected to a lesser extent, because interactions with the modified tube walls were shielded by the intervening active. However, interactions with the active molecules closer to the tube walls in turn slowed the active in the centre. The overall effect of these interactions was to lower D_{eff}.

It was flirter observed that sorption of guest molecules into the clay tubules arose either from the liquid or gas phases, with the degree of sorption depending on the local concentration/partial pressure of guests, and competition from other guest molecules.

Furthermore, a high degree of selectivity and release was able to be obtained through design of the host-guest interaction, such that only very specific classes of guest molecules were able be sorbed and released from the clay. Halloysite readily loads metal nitrates into the lumen 12, while organic compounds like benzophenone and 3-aminophenol were shown to only bind to the outer surface of the tubules (FIG. 2).

Modification of the various clay surfaces using differing surfactants revealed a high degree of selectivity for organic compounds. By varying the amount of surfactant used it was possible to alter the total uptake of guest molecules. Furthermore by varying the morphology of the clay tubule along with its modification it was possible to achieve a high selectivity in the uptake of guest molecules.

The organo-modification of a halloysite clay surface to a hydrophobic nature using quaternary amines was shown to load hydrophobic compounds like benzophenone and 3-aminophenol. This was particularly relevant for agrochemicals, as they too are hydrophobic. Hence the method enabled them to be sorbed into the lumen 12 of the modified halloysite tubules in a manner and to an extent never previously contemplated.

The selective uptake of guest molecules also enabled the release of the guest via a controlled method from the tubule. Variation in temperature, pressure and surface modification were shown to alter the release rates of the guest molecules from the tubules.

When applied in the field by conventional methods, agrochemicals are invariably subject to leaching, evaporation and degradation, each of which remove the active ingredients prematurely. Excess initial dosages are therefore necessary to compensate for the quick drop-off in levels associated with run-off or chemical breakdown. The controlled release technologies herein may avoid problems associated with the inefficiency and potential environmental consequences of conventional methods.

Further, the slow release of agrochemicals with a desired release over week and which is protected from runoff or chemical breakdown was therefore extremely beneficial to the currently applied excess overdosages. In addition, surface modification with highly favourable interactions for the target guest showed slow release compared to unfavourable interactions. This tweaking of favourable/unfavourable interactions with selective surface modification enabled slow and controlled release.

Materials could also be manufactured where controlled release can occur for more than one guest molecule at a time with competing release rates.

### Example 5

### Measurements

TEM work showed that not every active compound filled the tubes of unmodified halloysite. Generally the inorganic solids loaded into the lumen 12, while the organic compounds just adhered to the outside walls of the tubules. However, the conditioned clays herein showed remarkable loading for active organic compounds, and little to no loading for the inorganic compounds.

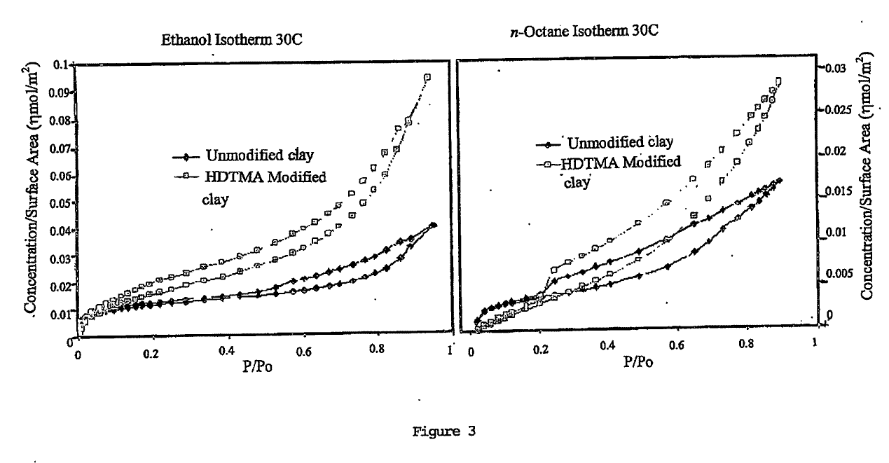

Water, ethanol and n-octane vapour sorption isotherms were measured using an Intelligent Gravimetric Analyzer (IGA) supplied by Hiden Analytical Ltd. The results are presented in FIG. 3.

The apparatus allowed vapour sorption isotherms and the corresponding kinetics of adsorption and desorption to be determined for individual pressure increments. The vapours used had varying hydrophobicity, and hence their affinity for sorption and release for the modified and unmodified clays was different.

More particularly, FIG. 3 shows the isotherms for n-octane and ethanol vapours at 30° C. for modified and unmodified halloysite, showing the amount sorbed for the available surface area.

Whilst modification of the clay was shown to reduce the lumen 12 volume which was expected, the total sorption capacity for the modified clay result might expect to be reduced. However, by looking at the available surface area, it was shown that sorption had been increased by 240% for ethanol and 170% for n-octane, while water sorption had rained the same.

Also, in the experiments HDTMA-modified halloysite was loaded variously with water, ethanol, n-octane, 3-aminophenol and benzophenone by way of illustration.

The tubular inorganic material is conditioned to allow loading with an active. The loaded active can comprise a waste or toxic compound which is loaded into the conditioned tubular inorganic material for the purpose of absorption and safe removal of the waste material. In this case the controlled release rate is extremely low and preferably the toxic or waste compound will be released only infinitesimally.

The use of conditioned tubular inorganic material such as modified halloysite to uptake and store toxic or waste products can be directed toward soil remediation. For example, in petrol stations volatile organic carbon (VOC) octane is deposited in soils. At present soils cannot be taken away from abandoned petrol station, due to high VOC contents. The soil is mixed with sand to aerate then left onsite for ten years. Removal of the VOC could be achieved by allowing the VOC to sorb to the modified halloysite to allow removal of the soil.

Further, greenhouse gases such as NOx vapours which are released by bacteria in soils can be sorbed by the modified halloysite. Halloysite-would then retard the NOx emissions from soils.

It should be noted that a much broader range of tubular inorganic minerals, surface modifications and load compounds can be employed. In summary:
1. Tubular inorganic materials included, but were not limited to the clays halloysite, imogolite, cylindrite and boulangerite.
2. Alternative surface modifications envisaged included variations of the surfactants used to bind to the clay surface to render the clay hydrophobic and therefore its affinity for target guest molecules over others (for example, by using alkylammonium (e.g. OTMA) phenylammonium (e.g. BTMA), substituted phenylammonium, alkylpyridinium, and phenylpyridinium ions as surface modifiers, together with mixtures thereof.
3. A large number of inorganic and organic compounds were shown to sorbed into the lumen 12. Other actives that may be loaded included, but were not limited to agrochemical (e.g. alochlor, metachlor, trifluralin), pharmaceuticals, biocides and antifoulants.

Foreseeable improvements to the degree of control over release rates included:
decreasing the cross-sectional area of the ends of the tubes with bulky anions attached under controlled pH conditions;
using knowledge of the sorption of hydrated metals to cap the tube ends with insoluble material;
capping the ends of the tubes with a capping material, the capping material having a low solubility, stability in uv radiation and decomposing under specific conditions. For example, a tetramethoxysilane-derived silica gel can be used as a capping material. The capping material allows the release to be triggered upon performance of a specific activation step which results in the decomposition of the capping;
coating the outside of the tubules with a porous polymer to further decrease release rates.

Such techniques promised to make these hosts suitable for longer-period release applications such as in anti-fouling paint formulations.

Other strategies and advantages for active (e.g. agrochemical) encapsulation within tubular clay materials such as halloysite involved:
the inclusion of insect pheromones into the pesticide formulations to attract insects to the point of release;
formulation of fibrous pellets for the controlled release of larger agents, for example biological systems such as spores;
weathering of spherical particles from the outside in, containing the guest trapped on the inside of the particle until the outside of the particle had weathered away—thus allowing controlled release through the gradual breakdown of the formulation. If the formulation separates into individual tubes when added to water, dispersion will comprise the spraying of individual tubules. Alternately, if the formulation does not separate into individual tubules the pellets themselves will be dispersed. In circumstances where the pellets are dispersed the weathering properties of the pellets will affect the release behaviour;
increased control over length distribution of tubes through appropriate sourcing, milling and/or centrifuging;
tubular clays providing a cylindrical lumen 12 into which to load additional active (cf. the active-carrying capacity of non-tubular clays (the % load) being limited to the surface area of the clay);
loading active into the lumen 12 to make the active less accessible, and reducing the level of exposure during handling and transport (cf. surface loading of flat clays not reducing the hazards of exposure to loaded active agents during handling);
release from the lumen 12 of clays being able to be further regulated by one-dimensional diffusion as the active escapes from its tubular container (cf. release from surface-loaded clays being limited to the rate of desorption of active);
modification of the inorganic tubes widening the range of hydrophobicities of compounds that can be loaded (cf. unmodified halloysite being hydrophilic, which limits its capacity to carry hydrophobic materials);
selection of modifying compounds (chemical agents) for optimum active/carrier interaction to allow further control over release rates (cf. unmodified halloysite presenting no carrier-specific control over the rate of diffusion out of the tube).

Whilst specific embodiments of the method of conditioning a tubular clay material, and of the material itself, have been described, it should be appreciated that the method and material can be embodied in many other forms.

1. A method of conditioning a tubular clay material to enable its loading with an active, the method comprising the step of exposing the tubular clay material to a chemical agent in a manner such that the agent sorbs to a surface of the clay material that is internal of the tube, the chemical agent being selected such that, when the agent is sorbed to the clay material internal surface, the affinity of the tubular clay material for the active is altered.

2. A method as claimed in claim 1, wherein the chemical agent is one or more of a surfactant, an alcohol and a phosphonate.

3. A method as claimed in claim 2, wherein, when the agent is a surfactant, the step of exposing the tubular clay material to the surfactant involves refluxing a solution of the material with the surfactant.

4. A method as claimed in claim 3, wherein the material is suspended in an aqueous solution with the surfactant and refluxed.

5. A method as claimed in claim 3, wherein the solution is refluxed at 80° C. for one hour.

6. A method as claimed in claim 3, wherein, after refluxing, the solution is cooled, filtered and washed to remove residual surfactant.

7. A method as claimed in claim 3, wherein the ratio (w/w) of tubular clay material to surfactant ranges from 1:1 to 1:20.

8. A method as claimed in claim 3, wherein the surfactant is a cationic surfactant selected from one or more of:
alkylammonium surfactants such as hexadecyl-trimethyl ammonium (HDTMA) and octyl-trimethylammonium (OTMA);
phenylammonium surfactants such as benzyl-trimethylammonium (BTMA) and phenyl-trimethylammonium (PTMA);
substituted phenylammonium surfactants;
alkylpyridinium surfactants;
phenylpyridinium surfactants.

9. A method as claimed in claim 2, wherein, when the agent is an alcohol, the step of exposing the tubular clay material to the alcohol involves mixing the clay material with the alcohol and heating the mixture so as to promote a condensation reaction between the alcohol and the clay material at the internal surface.

10. A method as claimed in claim 9, wherein the mixture is first heated using microwave irradiation and is subsequently refluxed.

11. A method as claimed in claim 10, wherein, during microwave irradiation and prior to refluxing, a vacuum is applied to the alcohol and clay material mixture to remove air from the clay material tubes.

12. A method as claimed in claim 10, wherein the alcohol is one that refluxes at a temperature just below its boiling point, and wherein the alcohol is first heated to its reflux temperature using the microwave irradiation.

13. A method as claimed in claim 10, wherein the alcohol is 1-octanol, and the mixture is heated to 194° C. using microwave irradiation and is then refluxed at 194° C. for 56 hours.

14. A method as claimed in claim 2, wherein, when the agent is a phosphonate, the step of exposing the tubular clay material to the phosphonate involves mixing the mixing the clay material with the phosphonate and heating the mixture so as to promote a reaction between the phosphonate and the clay material at the internal surface.

15. A method as claimed in claim 14, wherein the mixture is heated using microwave irradiation.

16. A method as claimed in claim 15, wherein, prior to heating the mixture using microwave irradiation, slow heating or vacuum cycling is applied to the phosphonate and clay material mixture to remove air from the clay material tubes.

17. A method as claimed in claim 14, wherein, after heating using microwave irradiation, the mixture is washed with dichloromethane and methanol to remove residual phosphonate.

18. A method as claimed in claim 14, wherein the phosphonate is a phosphonate ester such as diethyl phosphonate or diethyl benzyl-phosphonate.

19. A method as claimed in claim 1, wherein the tubular clay material is one or more of halloysite, imogolite, boulangerite and cylindrite.

20. A method as claimed in claim 1, wherein, after the tubular clay material is conditioned, it is subjected to loading with the active.

21. A method as claimed in claim 20, wherein the method of loading is an active-melting loading technique or an active-in-solution loading technique.

22. A method as claimed in claim 21, wherein, in the active-melting loading technique, the active and clay material are mixed and then heated to a temperature above the melting point for the active, and held there for a time period sufficient for the active to migrate into the tube.

23. A method as claimed in claim 22, wherein the time period is at least 5 hours.

24. A method as claimed in claim 21, wherein, in the active-in-solution loading technique, the active is dissolved in a solution in which it is soluble, and the clay material is added to this solution either with the active or after and stirred.

25. A method as claimed in claim 24, wherein the stirred solution is then subjected to ultrasound, subjected to a vacuum, centrifuged to remove supernatant solution, and then dried.

26. A method as claimed in claim 25, wherein the loaded clay material is dried at 90° C. for 24 hours.

27. A method as claim 20, wherein subsequent to loading with the active, the tube is capped with a capping material.

28. A method as defined in claim 27, wherein the capping material is a tetramethoxysilane-derived silica gel.

29. A method as claimed in claim 1, wherein the active can comprise one or more inorganic and organic chemicals, including mixtures thereof.

30. A method as claimed in claim 1, wherein the active is one or more of: an agrochemical, a pharmaceutical, a biocide, a bactericide, an anti-foulant, a cosmetic, a fragrance, a detergent, a hormone, a pheromone, a descaler, a cleaning agent, a corrosion inhibitor/preventer, an organic or inorganic pollutant or toxic material.

31. A method as claimed in claim 1, wherein the agrochemical is one or more of alochlor, metachlor and trifluralin.

32. A method as claimed in claim 1, wherein the affinity of the tubular clay material for the active is altered in a manner such that the material can act to release active in a controllable manner and/or to store or entrap active.

33. A method as claimed in claim 32, wherein the controlled release can be tuned through the selection of one or more chemical agents.

34. (canceled)

35. A method as claimed in claim 1, wherein the tube length and/or tube length distribution are varied prior to loading of chemical agent and active.

36. A method as claimed in claim 35, wherein the tube length and/or tube length distribution are varied by one or more of: tube sourcing control, milling and centrifuging.

37. (canceled)

38. A tubular clay material that has been conditioned to enable its loading with an active, the clay material comprising a chemical agent that is sorbed to a surface of the material that is internal of the tube and that alters the material's affinity for the active.

39. A tubular clay material as claimed in claim 38 that is as defined in, and as conditioned by exposing the tubular clay material to a chemical agent in a manner such that the agent sorbs to a surface of the clay material that is internal of the tube, the chemical agent being selected such that, when the agent is sorbed to the clay material internal surface, the affinity of the tubular clay material for the active is altered.

40. A tubular clay material as defined in claim 38, further comprising a capping material covering the ends of the tubular clay material.

41. A tubular clay material as defined in claim 41, wherein the capping material is a tetramethoxysilane-derived silica gel.
